(19) United States
(12) Patent Application Publication
(10) Pub. No.: US 2016/0114180 A1
(43) Pub. Date: Apr. 28, 2016

(54) ULTRATHIN MICROCHIP STRUCTURE

(71) Applicants: Jason TSENG, New Taipei City (TW); Harry WU, Taipei City (TW); Jing-Wen LIAO, New Taipei City (TW); Luca TSENG, New Taipei City (TW); Lucy TSENG, New Taipei City (TW)

(72) Inventors: Jason TSENG, New Taipei City (TW); Harry WU, Taipei City (TW); Jing-Wen LIAO, New Taipei City (TW); Luca TSENG, New Taipei City (TW); Lucy TSENG, New Taipei City (TW)

(21) Appl. No.: 14/681,815

(22) Filed: Apr. 8, 2015

(30) Foreign Application Priority Data
Oct. 23, 2014 (TW) 103218796

(51) Int. Cl. A61N 1/40

(52) U.S. Cl. CPC A61N 1/40

(57) ABSTRACT

An ultrathin microchip structure at least includes a flexible base material. The flexible base material includes an installation surface and a printed surface at an upper surface and a lower surface thereof, respectively. The installation surface is provided with one set or multiple sets of integrated circuits. Each of the integrated circuits at least includes a chip and at least one set of transceiver antenna. One or both of the installation surface and the printed surface of the flexible base material is/are applied with a nanometer film layer having characteristics of being waterproof, dustproof, wear resistant, and penetrable by the RF signal, thereby effectively simplifying the present invention as an ultrathin microchip.

### BACKGROUND OF THE INVENTION

a) Field of the Invention

The invention relates in general to an ultrathin microchip structure, and more particularly to an ultrathin microchip effectively simplified into a simple structure.

b) Description of the Prior Art

For body care purposes, modern people often use or wear healthcare supplies such as titanium necklaces, germanium bracelets, germanium titanium bracelets, magnetic ore products, charcoal products, and volcano ash products. It is claimed that benefits of promoting body health, and regulating physiological functions and body constitutions can be achieved after wearing the above supplies for an extended period of time. Most of the above body care supplies are passive products based on metal or magnetic materials. Instead of showing immediate effects, said products may only produce noticeable effects after extended periods of use or wear. Thus, certain users may become doubtful on the results or may be unable to wear these supplies for long-term, hence failing the claimed expected benefits.

The associated industrialists have developed a sheet-like biological resonance microchip including a printed circuit sticker. The printed circuit sticker is provided with at least one biological wave receiver, a high-frequency oscillation chip, and at least one biological microwave transmitter. The biological wave receiver, the high-frequency oscillation chip and the biological microwave transmitter are electrically connected to one another. As such, human biological wave characteristics from a human body are received by the biological wave receiver. These human biological waves are transmitted to the high-frequency oscillation chip which amplifies the power, and then fed back to the human body by the biological microwave transmitter. Thus, the main benefit of improving body health is accomplished with this chip by enhancing permanent resonance with the human body, using a concentrated bandwidth without producing any radioactive hazards and side effects.

The issue of associated conventional health care products producing noticeable effects after an extended period of use is solved by this microchip invention. As previously stated, the biological resonance chip receives human biological waves emitted from a human body, amplifies the waves, and then feeds the waves back to the human body for the use of the human body. When physiological functions of a human body are in an unsatisfactory condition, biological waves generated by the human body may be insufficient to power molecular and cellular functions. Such weak biological waves, however, are amplified by the high-frequency oscillation chip to a strength level desired by the human body to maximize its regulatory processes.

One concern is that the printed circuit sticker of the biological resonance chip lacks a wear protection layer that covers and protects the biological wave receiver, the high-frequency oscillation chip and the biological wave transmitter. On the other hand, if another cover layer is applied for extra protection, the overall thickness of the product may become excessive. The process of adding the additional layer may also damage the first layer or accidentally expose the interior structure to the exterior such that these components may become damaged.

### SUMMARY OF THE INVENTION

In view of the above drawbacks for current metalic/magnetic health product, the present invention is directed to an ultrathin microchip structure for improving those drawbacks of ease and length of use.

It is a primary objective of the present invention to provide an ultrathin microchip structure. In the microchip structure, a nanometer film layer of a flexible base material is applied to one or both of an upper surface and a lower surface to effectively provide a durable and convenient ultrathin microchip.

To achieve the above object, an ultrathin microchip structure according to an embodiment of the present invention includes a flexible base material and at least one set of integrated circuit. The flexible base material includes an installation surface and a printed surface. One or multiple sets of integrated circuits are provided on the installation surface. Each set of integrated circuits consists of a chip and a transceiver antenna. A major purpose of the present invention is that, at least one film layer is applied to one of the installation surface and the printed surface. The film layer has at least characteristics of being waterproof, dustproof, wear resistant, and penetrable by radio-frequency (RF) signals.

In one embodiment, the film layer may be transparent or semi-transparent.

In one embodiment, the chip is a signal generator adapted to generate RF signals.

In one embodiment, the transceiver antenna is adapted to receive an external magnetic field to provide the external magnetic field to the chip as a power source, and to transmit the RF signals generated by the chip to an exterior.

The above and other aspects of the invention will become better understood with regard to the following detailed description of the preferred but non-limiting embodiments. The following description is made with reference to the accompanying drawings.

### BRIEF DESCRIPTION OF THE DRAWINGS

FIG. 1 is a schematic diagram of an appearance of an ultrathin microchip structure of the present invention; and

FIG. 2 is an exploded view of an appearance of an ultrathin microchip structure of the present invention.

### DETAILED DESCRIPTION OF THE PREFERRED EMBODIMENTS

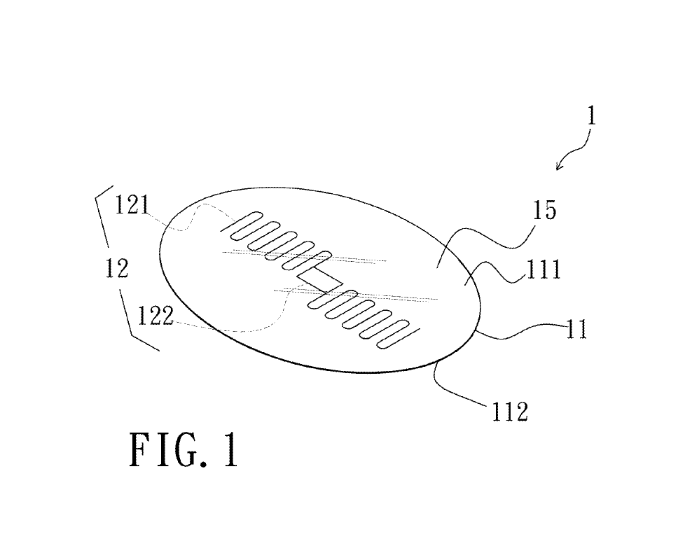

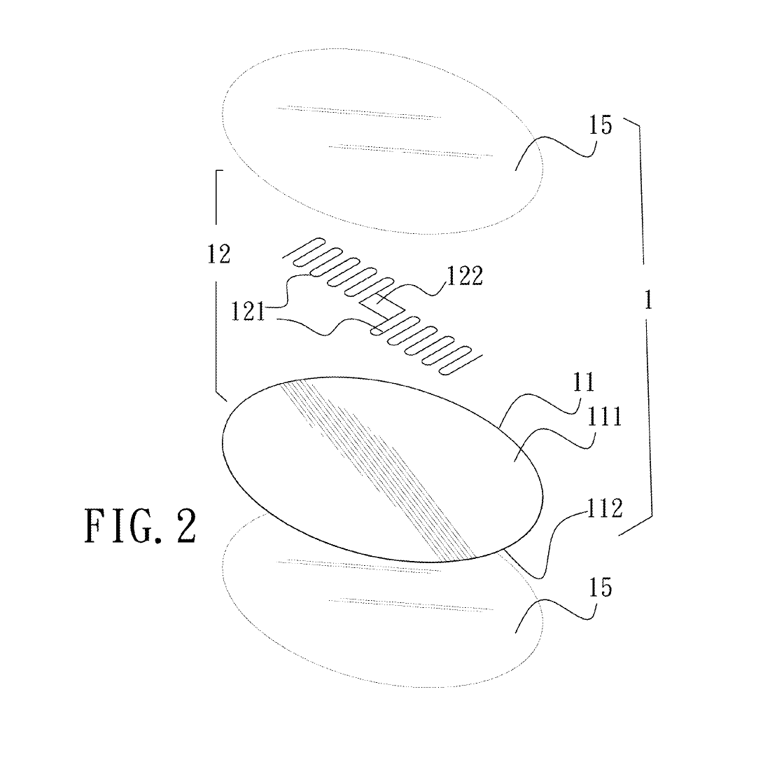

FIG. 1 and FIG. 2 show schematic diagrams of an appearance and an explode view, respectively, according to a first embodiment of the present invention.

As shown, an ultrathin microchip 1 of the present invention at least includes a flexible base material 11. For example, the flexible base material is formed from a PET (Polyethylene Terephthalate) or PVC (Polyvinyl Chloride) material, and may be in a size equivalent to that of a common card or a common piece of paper. The flexible base material 11 includes an installation surface 111 and a printed surface 112 at an upper surface and a lower surface, respectively. The installation surface 111 is provided with one set or multiple sets of integrated circuits 12.

A feature of the present invention is that, at least one nano-processed film layer 15 is evenly applied on the installation surface 111 and the printed surface 112. The film layer 15 has characteristics of being waterproof, dustproof, wear resistant, and penetrable by RF signals. Referring to FIG. 2, the film layer 15 is applied and covers a surface of the integrated circuit 12, so as to prevent various components (e.g., a chip 121 and a transceiver antenna 122) of the integrated circuit from damaged by external forces. In the present invention, the material and components of the film layer are not limited. Given that the characteristics of being waterproof, dustproof, wear resistant and penetrable by RF signals are achieved, any appropriate material (e.g., a film layer including a silicon compound or a fluoride compound) can be selected as the film layer 15 of the present invention.

Further, the integrated circuit 12 of the present invention mainly includes at least one chip 121 and at least one set of transceiver antenna 122. The chip 121 primarily serves a purpose as an RF signal generator. The transceiver antenna 122 is adapted to receive an external magnetic field and transmit wireless RF signals. A main operation principle of the integrated circuit 12 is that the external magnetic field (the earth's magnetic field) received by the transceiver antenna 122 is utilized as a permanent power source of the chip 121, which then generates wireless RF signals that are transmitted to a human body via the transceiver antenna 122. Accordingly, the wireless RF signals and bioelectricity of the human body generate a harmonic resonance procedure. Through the resonance procedure, effects of increasing body temperature, softening and dilating blood vessels, accelerating blood flow, promoting metabolism, enhancing body fitness, relaxing muscles, alleviating pains, relieving allergy, eliminating swollenness, reducing blood pressure, reducing anxiety, activating cells, promoting sleep, strengthening immune system, and boosting cardiovascular functions can be achieved for health well-being. In general, the integrated circuit 12 of the present invention is capable of generating the foregoing resonance procedure when located in proximity of about 2.5 cm from a human body. Therefore, the present invention may be installed in special garments including underwear, hats, eye shields, mouth masks, bras, wristbands, knee supporters, waist supporters, neck supporters, hoods, socks, shoes, and other clothing to generate the foregoing effects. In conclusion, the ultrathin microchip 1 of the present invention offers an effective ultrathin microchip structure as disclosed above.

While the invention has been described by way of example and in terms of the preferred embodiments, it is to be understood that the invention is not limited thereto. On the contrary, it is intended to cover various modifications and similar arrangements and procedures, and the scope of the appended claims, therefore, should be accorded the broadest interpretation so as to encompass all such modifications and similar arrangements and procedures.

What is claimed is:

1. An ultrathin microchip structure, comprising:
a flexible base material, having an installation surface and a printed surface, the installation surface being provided with one set or multiple sets of integrated circuits; and
at least one integrated circuit, each having at least one chip and at least one set of transceiver antenna;
wherein, one or both of the installation surface and the printed surface of the flexible base material is/are applied with at least one nanometer film layer penetrable by a radio-frequency (RF) signal.

2. The ultrathin microchip structure according to claim 1, wherein the chip serves as a signal generator adapted to generate the RF signal.

3. The ultrathin microchip structure according to claim 1, wherein the transceiver antenna is installed at the installation surface.

4. The ultrathin microchip structure according to claim 1, wherein the flexible base material is formed by a PET (Polyethylene Terephthalate) or PVC (Polyvinyl Chloride) material.
